(19) United States
(12) Patent Application Publication
(10) Pub. No.: US 2017/0172640 A1
(43) Pub. Date: Jun. 22, 2017

(54) LUNG CANCER TREATMENT WITH CONDENSABLE VAPOR AND THERAPEUTIC AGENT

(71) Applicant: Uptake Medical Technology Inc., Seattle, WA (US)

(72) Inventor: Erik Henne, Seattle, WA (US)

(21) Appl. No.: 15/377,017

(22) Filed: Dec. 13, 2016

Related U.S. Application Data
(60) Provisional application No. 62/268,297, filed on Dec. 16, 2015.
(60) Provisional application No. 62/268,288, filed on Dec. 16, 2015.

(51) Int. Cl. A61B 18/04; A61M 31/00

(52) U.S. Cl. CPC A61B 18/04; A61M 31/00; A61B 2018/048; A61B 2018/00541

(57) ABSTRACT

A method of treating a lung tumor or metastasis comprising forming a condensable vapor comprising water and a therapeutic agent and delivering the vapor and therapeutic agent to a target tissue in a region of a patient's lung. The therapeutic agent may be chemotherapy drug. In embodiments, vapor ablation is combined with systemic therapy.

### CROSS-REFERENCE TO RELATED APPLICATIONS

This application claims priority to and the benefit of Provisional Application No. 62/268,297, field Dec. 16, 2015, entitled “LUNG CANCER TREATMENT WITH CONDENSABLE VAPOR AND THERAPEUTIC AGENT” and Provisional Application No. 62/268,288, filed Dec. 16, 2015, and entitled “VAPOR ABLATION OF TUMORS TO INCREASE IMMUNE RESPONSE”, each of which is incorporated by reference in its entirety.

### BACKGROUND OF THE INVENTION

Condensable vapor has been used to treat lung conditions, such as emphysema, COPD, lung tumors, etc., by delivering heat from the condensable vapor to lung tissue to ablate, shrink or otherwise alter the tissue. See, e.g., U.S. Pat. No. 7,913,698; U.S. Pat. No. 7,993,323; US Publ. No. 2013/0267939 and US Publ. No. 2015/0094607. U.S. Pat. No. 7,913,698 discloses the delivery of microparticulates to promote fibrosis or a painkiller along with the vapor.

US Publ. No. 2003/0199449 discloses the combination of localized drug delivery along with RF ablation to treat tumors. This publication does not describe the use of condensable vapor as the ablation modality, however, nor does it describe the treatment of lung tumors with the ablation/drug delivery combination.

Ito, F., et al., “Immune Adjuvant Activity of Pre-Resectional Radiofrequency Ablation Protects against Local and Systemic Recurrence in Aggressive Murine colorectal Cancer,” PLOS One (Nov. 23, 2015), describes the benefits of RF ablation of tumors when combined with resection as compared to resection alone or RF ablation alone. This article does not address the synergies of combining vapor ablation of tumors with other tumor treatments.

Other publications include: K. F. Chu and D. E. Dupuy, “Thermal ablation of tumours: biological mechanisms and advances in therapy,” Nat. Rev. Cancer, vol. 14, no. 3, pp. 199-208, 2014; M. S. Sabel, “Cryo-immunology: A review of the literature and proposed mechanisms for stimulatory versus suppressive immune responses,” Cryobiology, vol. 58, no. 1, pp. 1-11, 2009; W. Rao, Z.-S. Deng, and J. Liu, “A review of hyperthermia combined with radiotherapy/chemotherapy on malignant tumors.,” Crit. Rev. Biomed. Eng., vol. 38, no. 1, pp. 101-16, 2010; Solazzo, M. Ahmed, R. Schor-Bardach, W. Yang, G. D. Girnun, S. Rahmanuddin, T. Levchenko, S. Signoretti, D. R. Spitz, V. Torchilin, and S. N. Goldberg, “Liposomal doxorubicin increases radiofrequency ablation-induced tumor destruction by increasing cellular oxidative and nitrative stress and accelerating apoptotic pathways.,” Radiology, vol. 255, no. 1, pp. 62-74, 2010; W. Yang, M. Ahmed, M. Elian, E.-S. a Hady, T. S. Levchenko, R. R. Sawant, S. Signoretti, M. Collins, V. P. Torchilin, and S. N. Goldberg, “Do liposomal apoptotic enhancers increase tumor coagulation and end-point survival in percutaneous radiofrequency ablation of tumors in a rat tumor model?,” Radiology, vol. 257, no. 3, pp. 685-696, 2010; Schueller 2004 Int J Oncology “Heat shock protein expression induced by percutaneous radiofrequency ablation of hepatocellular carcinoma in vivo”; K. Hiroishi, J. Eguchi, T. Baba, T. Shimazaki, S. Ishii, A. Hiraide, M. Sakaki, H. Doi, S. Uozumi, R. Omori, T. Matsumura, T. Yanagawa, T. Ito, and M. Imawari, “Strong CD8+ T-cell responses against tumor-associated antigens prolong the recurrence-free interval after tumor treatment in patients with hepatocellular carcinoma,” J. Gastroenterol., vol. 45, no. 4, pp. 451-458, 2010; G. Gravante, G. Sconocchia, S. L. Ong, A. R. Dennison, and D. M. Lloyd, “Immunoregulatory effects of liver ablation therapies for the treatment of primary and metastatic liver malignancies.,” Liver Int., vol. 29, no. 1, pp. 18-24, 2009; M. H. Ravindranath, T. F. Wood, D. Soh, A. Gonzales, S. Muthugounder, C. Perez, D. L. Morton, and A. J. Bilchik, “Cryosurgical ablation of liver tumors in colon cancer patients increases the serum total ganglioside level and then selectively augments antiganglioside IgM.,” Cryobiology, vol. 45, no. 1, pp. 10-21, 2002; and Andrew B. Sharabi, et al., “Stereotactic Radiation Therapy Combined With Immunotherapy: Augmenting the Role of Radiation in Local and Systemic Treatment,” Oncology Journal, May 15 2015.

Notwithstanding the above, an improved method and systems for treating lung tumors and metastases still exist.

### SUMMARY OF THE INVENTION

A method of treating a lung tumor or metastasis includes forming a condensable vapor comprising water and a therapeutic agent and delivering the vapor and therapeutic agent to a target tissue in a region of a patient's lung. The therapeutic agent may be a chemotherapy drug.

In embodiments a method of treating a lung tumor or metastasis in a patient includes delivering a condensable vapor to a target tissue in a region of a patient's lung and providing an additional therapy to treat the tumor or metastasis.

In embodiments the additional therapy includes delivery of a chemotherapy drug, an immunotherapy agent, or a checkpoint inhibitor. Delivery may be carried out either systemically, locally, or in combinations thereof.

In embodiments the method includes condensing the vapor to ablate the target tissue.

In embodiments the method includes stimulating an immune response with the condensable vapor.

Still other descriptions, objects and advantages of the present invention will become apparent from the detailed description to follow, together with the accompanying drawings.

### BRIEF DESCRIPTION OF THE DRAWINGS

FIG. 1 is an illustration depicting the formation and delivery of vaporized water and a therapeutic agent to a lung region in accordance with an embodiment of the invention.

FIG. 2 is a flow chart depicting a method 100 for treating a tumor or metastases in the lung region.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All publications and patent applications mentioned in this specification are incorporated herein by reference in their entirety to the same extent as if each individual publication or patent application was specifically and individually indicated to be incorporated by reference.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Described herein are methods for treating lung tumors comprising forming a condensable vapor and delivering the condensable vapor to the lung region where the tumor is located. In embodiments of the invention, the condensable vapor is enhanced with a therapeutic agent, and in other embodiments, the condensable vapor supplements a systemic or local treatment.

Therapeutic Agent Enhanced Vapor Ablation

As described herein, in embodiments of the invention, a tumor or metastasis treatment method involves the delivery of drugs (such as, for example, and without limitation, chemotherapy drugs, immunotherapy agents and/or checkpoint inhibitor drugs) to a region of a lung via condensable vapor. The tissue changes caused by the heat released by the vapor during condensation serves to lower the amount of the drug or other agent that would otherwise be required to treat the tumor or metastasis, thereby, e.g., lowering the toxicity of the chemotherapy drug or increasing the immune response to the chemotherapy drug or immunotherapy agent. Vapor delivery can be targeted to only to the region of interest within the lung, and the vapor will carry the drug or other agent throughout the lung region to ensure complete distribution of the drug or agent. In addition, the efficacy of some drugs can be improved by delivering the drugs to heated tissue.

Embodiments described herein include treating the patient at different stages of the disease. For example, in early stage lung cancer, local ablation of the tumor(s) with condensable vapor combined with drug delivery by the vapor addresses indolent cancer cells with minimal impact. In late stage lung cancer, targeted delivery of a chemotherapy drug to the region of the tumor(s) serves to reduce the amount of chemotherapy drug delivered to the patient systemically. Finally, in lung metastases as well as in other lung cancers, localized drug delivery via condensable vapor combined with targeted ablation serves to improve survival by providing higher efficacy and lower toxicity.

In embodiments, use of condensable vapor for tissue heating avoids the tissue desiccation and thermal fixation caused by other thermal ablation modalities, thereby increasing the efficacy of the therapy. Also, vapor delivery is targeted to the region of interest through use of a vapor delivery catheter as described herein. In addition, since it follows the anatomic boundaries of the airway, the effect of the condensable vapor is limited to the tissue of interest within the lung.

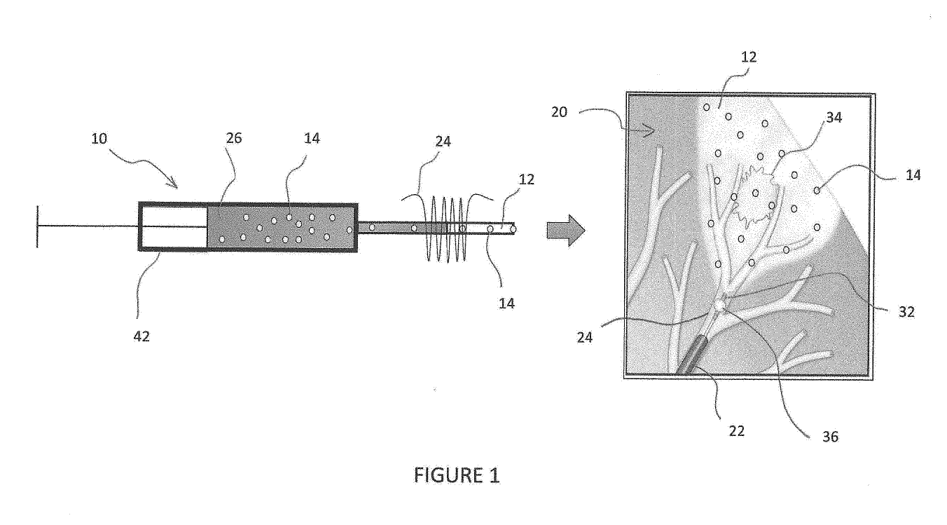

FIG. 1 shows a vapor delivery apparatus 10 and the delivery of vaporized water 12 and a therapeutic agent 14 (such as, e.g., a chemotherapy drug) to a lung region 20 via a vapor delivery catheter 22 advanced into the lung airway 24. In particular, the vaporized water 12 and a therapeutic agent 14 is shown exiting tube 32 towards tumor 34. An occluding member 36 (e.g., an inflatable balloon) is shown in an expanded state to fluidly isolate the targeted lung region.

In this embodiment, the combination of liquid water 26 and therapeutic agent 14 is heated to 100° C. or higher by an RF inductive heating coil 24. The vapor and therapeutic agent mixture flows through the catheter 22 to the target tissue. A syringe 42 is shown holding the liquid water and to drive the liquid to the target tissue. However, the catheter structure, pump and delivery, heating, and control systems may vary widely. An example of another system to deliver a condensable vapor to the tissue is described in US Publ. No. 2016/0220297.

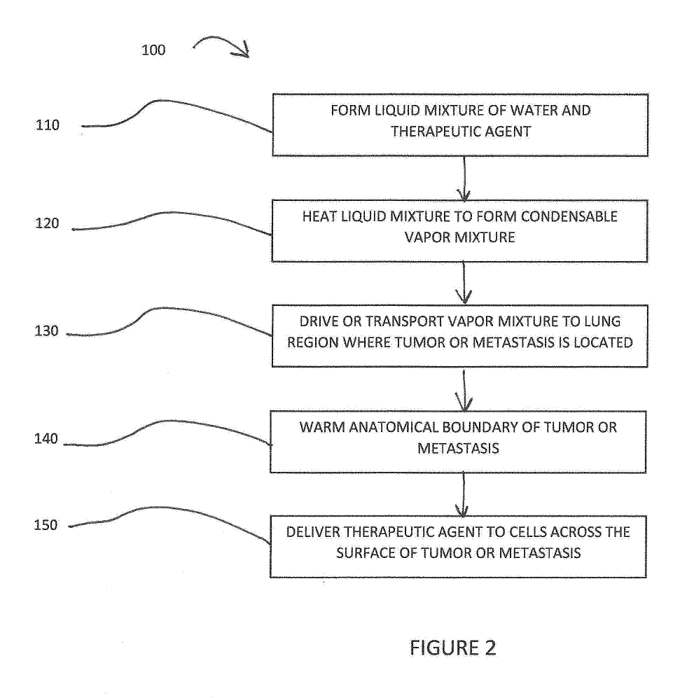

FIG. 2 shows a method 100 for treating a tumor or metastasis in the lung. Step 110 states to form liquid mixture of water and a therapeutic agent. Optionally, this step can include selecting applicable or useful therapeutic agents for treating cancer based on an agent's known effectiveness for treating cancer. The method may also include the step of excluding agents that are inoperable in vapor form. The method may also include the step of excluding agents that are inoperable at temperatures near or at 100 degrees Celsius.

The therapeutic agent and water may be mixed. The therapeutic agent and water may be warmed to facilitate uniform dispersion of the therapeutic agent in the water. Examples of mixtures include suspensions, colloids, or solutions.

Concentrations of the agent to water may vary. In embodiments the concentration of the therapeutic agent is based on the therapeutic dosage of the agent for its intended use to treat similar sized tumors, and in view of the size of lung tumor to be treated, or volume of the lung region to be filled with the vapor mixture.

Step 120 states to heat the liquid mixture to form a condensable vapor mixture. Heating or vaporizing the liquid mixture may be carried out as described herein. Exemplary heating techniques include induction, resistive heating, and boiling.

Step 130 states to drive or transport the vapor mixture to the lung region where the tumor or metastasis is located. As described above in connection with the embodiment shown in FIG. 1, a syringe may drive the mixture to the lung region. However, pumps, and other systems and means including pressurized systems may drive the fluid through the delivery apparatus and to the target region.

Step 140 states to warm the anatomical boundary of the tumor or the metastasis. As described herein, this step sends the warm vapor mixture across the margin or boundary of the tumor. Unlike a surgical instrument requiring contact to deliver heat (e.g., RF ablation catheter) directly to the tumor, the subject invention drives a vapor mixture into the target airways, filling the space surrounding the tumor, thereby naturally or automatically following the boundary of the tumor.

Step 150 states to deliver the therapeutic agent to cells across the surface of the tumor or metastasis. As described herein, the vapor to contact the tumor boundary, warms the cells, and facilitates delivering the drug into the cells, and or stimulate the immune response.

It should be understood, however, that steps of the method described in FIG. 2, and other method steps described herein, are intended to be combinable in any logical sequence except where such steps are exclusive to one another.

Systemic Treatment Enhanced with Vapor Ablation

In embodiments a tumor or metastasis treatment method combines the vapor ablation of a tumor with one or more other tumor treatment therapies, such as resection, immunotherapy or chemotherapy. The tissue changes caused by the heat released by the vapor during condensation serve to lower the amount of the drug or other agent that would otherwise be required to treat the tumor or metastasis, thereby, e.g., lowering the toxicity of the chemotherapy drug or increasing the immune response to the chemotherapy drug or immunotherapy agent.

For example, localized vapor ablation of a lung tumor or lung metastasis serves to provide a synergistic effect when combined with localized or systemic delivery of a chemotherapy drug by providing higher drug efficacy and/or permitting a lower drug dose. In addition, vapor ablation of a lung tumor or lung metastasis serves to increase the patient's immune response via the abscopal effect, thereby improving the efficacy of surgical resection of that tumor or other lung tumors and improving the efficacy of any local or systemic immunotherapy provided to the patient.

The use of condensable vapor for tissue heating avoids the tissue desiccation and thermal fixation caused by other thermal ablation modalities, thereby increasing the efficacy of the therapy. Also, vapor delivery serves to target the region of interest through use of a vapor delivery catheters as described herein. In addition, because the condensable vapor follows the anatomic boundaries of the airway, the effect of the condensable vapor is desirably limited to the tissue of interest within the lung.

Embodiments described herein include treating the patient at different stages of the disease. For example, in early stage lung cancer, even after lobectomy, there is up to 30% recurrence rate. Local ablation and systemic therapy, in combination, and as described herein, is performed to address indolent cancer cells with minimal impact. In late stage lung cancer, necessary therapy can be too toxic for the patient. Methods described herein include targeted local drug delivery with vapor ablation. In lung metastases, wherein the initial or primary cancer is well managed, the lung metastases are treated with methods described herein. In embodiments methods include personalized management of local ablation and systemic therapy serves to improve survival by higher efficacy and lower toxicity.

Ablation and Chemotherapy

In embodiments local vapor ablation is combined with chemotherapy for synergistic effects. In one embodiment, ablation is combined with nanoparticle chemotherapy. Apoptotic activity is accelerated. A benefit of combining vapor ablation with chemotherapy is systemic toxicities can be kept low with combined effect. Another benefit is a number of chemotherapy drugs are more effective when the tissue is heated. In embodiments, a method includes applying a local hyperthermia and chemotherapy. In another embodiment, a method includes warming the cell membranes of the target tissue without ablating the tissue and delivering the drugs through the cell membrane. In another embodiment, the method includes reducing the interstitial fluid pressure by delivering condensable vapor to the target tissue area, and delivering drugs through the cell membrane.

Ablation and Immunotherapy

In embodiments local vapor ablation is combined with immunotherapy, serving to induce an anti-tumor immune response—abscopal effect or “in vivo vaccine” effect.

In embodiments, ablation is applied to induce the Heat Shock Proteins (HSP), which are involved in various immunological processes, stimulate immune system, and thus increase survival.

In embodiments, ablation is applied to the target tissue to release antigens, stimulate immune system, and thus increase survival. For example, in embodiments, ablation is applied to inhibit tumor dependent T cell inactivation at ablation margins. Additionally, in other embodiments, the immune system is stimulated from ablation (e.g., local vapor ablation) and checkpoint inhibitor drugs are delivered, serving to reduce recurrence.

Embodiments described herein have a number of advantages. Some of the advantages arise from the unique energy type employed in the subject invention methods. For example, in embodiments, vapor thermal energy is applied such that it does not induce desiccation or thermal fixation. In embodiments, vapor thermal energy is applied to the tissue such that the tissue architecture remains intact and the therapeutic injury is not seen as a “foreign body”.

In embodiments, a method includes injuring or causing the ablation on the cell surface of the target, serving to induce apoptosis or cause a “disruptive necrosis.” The method further comprises leaving intact tumor-specific antigens in situ serving to stimulate the immune response.

In embodiments, a method includes applying vapor thermal energy to heat the target tissue surface but not induce coagulative destruction of organelles; to minimize the denaturing of proteins; and to maximize spill of recognizable intracellular products into system circulation. The above steps serve to provide higher rates of tumor local control and reduce recurrence.

In embodiments, a method includes constraining energy and vapor ablation to follow anatomical margins of the target tissue.

In embodiments, a method includes applying vapor ablation to a lung tumor in combination with another local or systemic therapy including, e.g., one or more of the therapies described herein. In embodiments, the condensable vapor is applied to the boundary of the tumor. The condensable vapor heats the exterior of the tumor sufficient to commence cell death but to not coagulate or destroy the tissue architecture.

In embodiments, the condensable vapor to contact the anatomical boundary of the tumor has a temperature of about 100 degree C.

Although a number of embodiments have been disclosed above, it is to be understood that other modifications and variations can be made to the disclosed embodiments without departing from the subject invention.

1. A method of treating a lung tumor or metastasis comprising forming a condensable vapor comprising water and a therapeutic agent and delivering the vapor and therapeutic agent to a target tissue in a region of a patient's lung.

2. The method of claim 1 further comprising condensing the vapor to ablate the target tissue.

3. The method of claim 1 further comprising stimulating an immune response with the condensable vapor.

4. The method of claim 1 wherein the therapeutic agent is a chemotherapy drug.

5. The method of claim 1 wherein the therapeutic agent is an immunotherapy agent.

6. The method of claim 1 wherein the therapeutic agent is a checkpoint inhibitor.

7. A method of treating a lung tumor or metastasis comprising warming an anatomical margin of the lung tumor or metastasis with a condensable vapor mixture wherein the vapor mixture comprises a therapeutic agent.

8. The method of claim 7 further comprising heating cells on the anatomical margin to a threshold temperature, wherein the threshold temperature is less than the temperature for inducing tissue coagulation and re-architecture.

9. The method of claim 8 wherein the threshold temperature is less than or equal to 100 degrees Celsius.

10. The method of claim 7 further comprising injuring but not killing cells on the anatomical margin with the vapor mixture and stimulating an immune response against the lung tumor or metastasis.

11. A method of treating a lung tumor or metastasis comprising delivering a condensable vapor to a target tissue in a region of a patient's lung and providing an additional therapy to the patient for treatment of the tumor or metastasis.

12. The method of claim 11 further comprising condensing the vapor to ablate the target tissue.

13. The method of claim 11 further comprising stimulating an immune response with the condensable vapor.

14. The method of claim 11 wherein the additional therapy is delivery of a chemotherapy drug.

15. The method of claim 11 wherein the additional therapy is delivery of an immunotherapy agent.

16. The method of claim 11 wherein the additional therapy is delivery of a checkpoint inhibitor.

17. The method of claim 11 wherein the additional therapy comprises systemic delivery of a therapeutic agent.

18. The method of claim 17 wherein the therapeutic agent is one agent selected from the group consisting of a chemotherapy drug, immunotherapy agent, and checkpoint inhibitor.

19. The method of claim 11 wherein the additional therapy comprises local delivery of a therapeutic agent.

20. The method of claim 19 wherein the therapeutic agent is a chemotherapy drug.
